(19) United States
(12) Patent Application Publication
(10) Pub. No.: US 2023/0007888 A1
(43) Pub. Date: Jan. 12, 2023

(54) DUAL-CORE ABSORBENT SANITARY ARTICLE AND A METHOD FOR PRODUCING THE SAME

(71) Applicant: Fameccanica.Data S.p.A., San Giovanni Teatino (Chieti) (IT)

(72) Inventors: Gabriele SABLONE, San Giovanni Teatino (Chieti) (IT); Guido BONELLI, San Giovanni Teatino (Chieti) (IT)

(21) Appl. No.: 17/861,340

(22) Filed: Jul. 11, 2022

(30) Foreign Application Priority Data
Jul. 12, 2021 (EP) 21184976.5

(51) Int. Cl. A61F 13/511; A61F 13/15

(52) U.S. Cl. CPC A61F 13/5116; A61F 13/15699

(57) ABSTRACT

An absorbent sanitary article includes a fluff absorbent core having at least one through aperture, a fluff-free absorbent core, and an acquisition and diffusion layer set between the fluff absorbent core and the fluff-free absorbent core and overlapping the at least one through aperture.

### FIELD OF THE INVENTION

The present invention relates to absorbent sanitary articles, such as disposable diapers, incontinent articles, and the like, having a dual-core absorbent structure.

The present invention also relates to a method for producing such absorbent sanitary articles.

### PRIOR ART

Absorbent articles such as disposable diapers, adult incontinent pads, and the like are manufactured in many different sizes and shapes. Incontinent pads are disposable absorbent articles worn by incontinent persons between the legs and fastened around the waist of the wearer.

An absorbent sanitary article typically has a structure that comprises a central body or chassis having a front section and a rear section. The chassis normally includes a permeable topsheet intended to come into contact with the user's skin when the article is worn, an impermeable backsheet and an absorbent core sandwiched between the topsheet and the backsheet. The front and rear sections of the chassis are normally closed around the user's waist by means of hook-and-loop fasteners, better known as Velcro© fastening devices.

There is a constant effort to make such absorbent articles more comfortable and discrete and less obtrusive for the wearer. This is particularly true in the case of adult incontinent pads. Adult incontinent pads, by their nature, are quite thick so they can handle the relatively large quantities of urine they receive. The desire for less bulky incontinent pads is particularly great among incontinent persons, many of whom are elderly adults, because it can be embarrassing to wear diaper-like articles. In addition, most individuals would rather use some type of article that could be worn with the undergarments they normally wear.

One key component of an absorbent article that contributes to its size and bulk is the absorbent structure used therein.

The absorbent core of the absorbent sanitary article ideally should be such that the absorbent article absorbs liquid exudates immediately when they are discharged so that such exudates will not pool or otherwise remain on top of the core.

The absorbent core ideally should also be constructed so exudates initially absorbed will be immediately transported to a place within the absorbent core where they can be stored. The absorbent core should also provide a system of distribution and storage for exudates that efficiently uses the entire capacity of the absorbent core. One problem that often arises in absorbent articles without such a system (particularly those absorbent articles that use the same layer or batt of material to serve the different functions of acquiring, distributing, and storing exudates) is that the storage capacity of the absorbent article is exhausted prematurely. This can occur in several different ways. In many absorbent articles, the saturation of the absorbent material in the region where exudates are initially deposited reduces the ability of the absorbent material to transport additional exudates to other parts of the core. This phenomenon can also lead to the undesirable pooling of exudates on top of the core discussed above.

### OBJECT AND SUMMARY OF THE INVENTION

The object of the present invention is to provide an absorbent sanitary article with an improved shape that has an absorbent structure that quickly acquires and distributes exudates throughout the absorbent core where they can be stored.

An object of the present invention is to provide an absorbent article, such as an adult incontinent pad, having a rapid acquiring, multiple layer absorbent structure having an improved shape that can be worn in the wearer's usual undergarments.

Another object of the present invention is to provide an absorbent structure for an absorbent sanitary article which is especially efficient in acquiring, distributing, and storing liquid exudates as they are deposited on the absorbent article.

In particular, an object of the present invention is to provide an absorbent structure in which the functions of acquiring and distributing exudates are handled by layers that are separate from the layers used for storing exudates.

According to the present invention, these objects are achieved by an absorbent sanitary article having the features of claim 1.

According to another aspect, the invention relates to a method for manufacturing absorbent sanitary articles having the features of claim 13.

The claims form an integral part of the technical disclosure provided here in relation to the invention.

### BRIEF DESCRIPTION OF THE DRAWINGS

The present invention will now be described with reference to the attached drawings, provided purely by way of non-limiting example, wherein:

FIG. 1 is a schematic plan view showing an absorbent sanitary article according to the present invention in an extended position,

FIG. 2 is a schematic cross-section taken along the line II-II of FIG. 1, and

FIG. 3 is a schematic side view of an embodiment of a machine for manufacturing absorbent sanitary articles according to the present invention.

It should be appreciated that the attached drawings are schematic and not to scale with respect to real products. Various figures may not be represented in the same scale. Also, in various figures some elements may not be shown to better show other elements.

### DETAILED DESCRIPTION

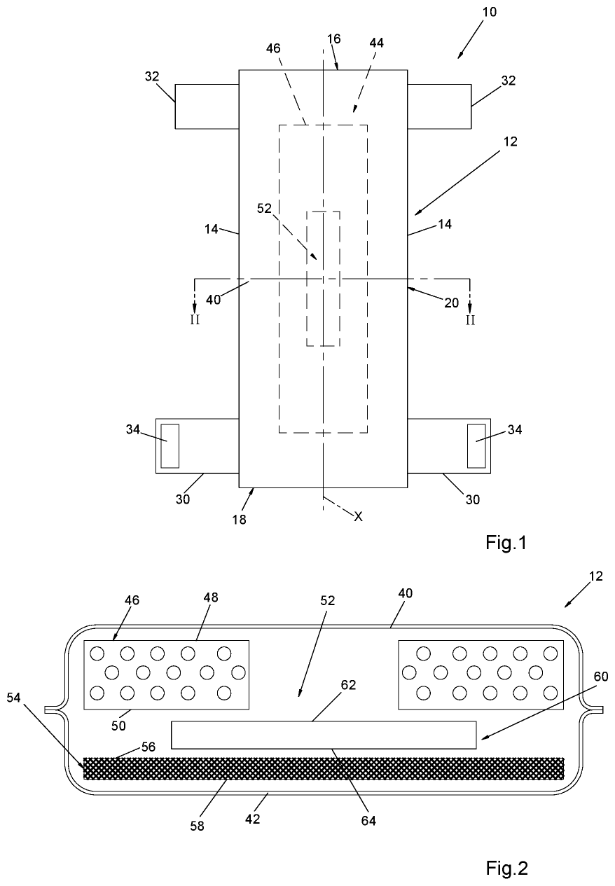

With reference to FIG. 1, numeral 10 indicates an absorbent sanitary article according to an embodiment of the present invention.

The absorbent sanitary article 10 comprises a chassis 12 elongated along a longitudinal axis X. The chassis 12 has two side edges 14, a front section 16 and a rear section 18. The front section 16 and the rear section 18 in use are closed around the user's waist. A crotch section 20 extends between the front section 16 and the rear section 18. In use, the crotch section 20 is arranged between the legs of the user.

The absorbent sanitary article 10 comprises a fastening device configured for fastening the front section 16 and the rear section 18 to each other. The fastening device may comprise two back side panels 30 attached to the rear section 18 of the chassis 12 and two front side panels 32 attached to the front section 16, extending laterally beyond respective side edges 14. The back side panels 30 and/or the front side panels 32 may be elastically stretchable in a transverse direction. The back side panels 30 may have distal portions provided with micro-hook elements 34 which form a releasable surface connection with a micro-loop layer provided on the front side panels 32.

With reference to FIG. 2, the chassis 12 comprises a topsheet 40 made of a permeable material which, in use, is in contact with the user's skin, an impermeable backsheet 42 which, in use, is in contact with the user's undergarments, and an absorbent structure 44 arranged between the topsheet 40 and the backsheet 42. The topsheet 40 and the backsheet 42 are joined to each other, e.g. by welding or by glue, around the absorbent structure 44 along the side edges 14 and transverse edges of the chassis 12.

The absorbent structure 44 comprises a fluff absorbent core 46 adjacent to the topsheet 40 and comprising a mix of cellulose fluff and superabsorbent granular material dispersed in the cellulose fluff.

The fluff absorbent core 46 is a soft pad having a generally flat shape elongated in the longitudinal direction X with two opposite first and second surfaces 48, 50. The first surface 48 may be in contact with the topsheet 40. The fluff absorbent core 46 has at least one through aperture 52 which extends through the mix of cellulose fluff and superabsorbent granular material between the two opposite surfaces 48, 50. In a possible embodiment the through aperture 52 is shaped as a channel (see FIG. 1) elongated along the longitudinal axis X. The fluff absorbent core 46 may be enclosed in a thin permeable non-woven layer which contains the mix of cellulose fluff and superabsorbent granular material.

The absorbent structure 44 comprises a fluff-free absorbent core 54 adjacent to the backsheet 42. The fluff-free absorbent core 54 comprises a plate-shaped non-woven web containing high quantity of superabsorbent granular material dispersed in the non-woven web.

The fluff-free absorbent core 54 has two opposite third and fourth surfaces 56, 58, generally parallel to the first and second surfaces 48, 50 of the fluff absorbent core 46. The fourth surface 58 may be in contact with the backsheet 42.

The fluff-free absorbent core 54 has a thickness which is considerably less than the thickness of the fluff absorbent core 46. In a possible embodiment, the thickness of the fluff-free absorbent core 54 is comprised between 1 and 4 mm, preferably between 1 and 2 mm.

The fluff-free absorbent core 54 comprises a non-woven material formed by processes such as spunbonding, meltblowing, carding, and the like. Non-limiting examples of suitable fibers include spunbond, spunlaid, meltblown, spunmelt, solvent-spun, electrospun, carded, film fibrillated, melt-film fibrillated, air-laid, dry-laid, wet—laid staple fibers, and other non-woven web materials formed in part or in whole of polymer fibres as known in the art, and workable combinations thereof. Nonwovens do not have a woven or knitted filament pattern. It is to be appreciated that non-wovens having various basis weights can be used. For example, some nonwovens may have a basis weight from about 5 gsm to about 50 gsm, preferably between 8 and 30 gsm.

In a possible embodiment, the fluff-free absorbent core 54 may comprise at least one layer of high loft non-woven material. An high loft non-woven is an ATB (air through bond) in two-component fibres, with high apparent thickness and large gaps between the fibers to accommodate the SAP. The weight range of high loft non-woven webs is comprised between 20 to 150 gsm, preferably between 30 to 80 gsm.

In a possible embodiment, the fluff-free absorbent core 54 comprises super-absorbent granular material dispersed in the non-woven fibres in an amount comprised between 100 to 800 gsm, preferably between 200 and 600 gsm. The fluff-free absorbent core 54 may be manufactured as disclosed in IT102020000019960 by the same Applicant.

In a possible embodiment, the fluff-free absorbent core 54 may be enclosed in a liquid permeable wrapping layer. In a possible embodiment, the fluff-free absorbent core 54 may comprise upper and lower non-woven layers. In a possible embodiment, both upper and lower non-woven layers contain super-absorbent granular material. In a possible embodiment, only the lower non-woven layer contains super-absorbent granular material. In both cases, the fluff-free absorbent core 54 may be enclosed in a liquid permeable wrapping layer.

The absorbent structure 44 comprises an acquisition and diffusion layer (ADL) 60 set between the fluff absorbent core 46 and fluff-free absorbent core 54 and overlapping the at least one through aperture 52 of the fluff absorbent core 46. The acquisition and diffusion layer 60 may be formed by a high loft non-woven material, which does not contain neither cellulose fluff nor superabsorbent polymers. The acquisition and diffusion layer 60 has the shape of a thin plate having opposite fifth and sixth surfaces 62, 64 generally parallel to the surfaces 48, 50 and 56, 58 of the fluff absorbent core 46 and fluff-free absorbent core 54. The fifth and sixth surfaces 62, 64 of the acquisition and diffusion layer 60 are in contact, respectively, with the second surface 50 of the fluff absorbent core 46 and with the third surface 56 of the fluff-free absorbent core 54.

The fifth surface 62 of the acquisition and diffusion layer 60 covers at least the portion of the second surface 50 of the fluff absorbent core 46 on which the at least one aperture 52 extends. The sixth surface of the acquisition and diffusion layer 60 is 30% or more of the third surface 56 of the fluff-free absorbent core 54. The acquisition and diffusion layer 60 may have a mono-layer or a multi-layer structure.

Normally a fluff-free absorbent core has liquid acquisition times longer than a fluff absorbent core. In the arrangement according to the invention, the acquisition and diffusion layer 60 acquires rapidly a surge of liquid, such as urine, passing through the through aperture 52 of the fluff absorbent core 46 and serves as a temporary reservoir for the liquid until the fluff-free absorbent core 42 can absorb the liquid. Also, the acquisition and diffusion layer 60 receives the liquid concentrated mainly in the area facing the through aperture 52 and distributes the liquid over a larger surface to facilitate absorption of the liquid by the fluff-free absorbent core 54. The acquisition and diffusion layer 60 forms a spacer between the fluff absorbent core 46 and the fluff-free absorbent core 42 and allows the liquid passing through the aperture 52 to be distributed over most of the surface of the fluff-free absorbent core 42.

Traditional dual-core absorbent sanitary articles have a very high absorbing capacity but are bulky and obtrusive for the wearer.

The absorbent sanitary article according to the present invention retains the same very high absorbing capacity of prior art dual-core absorbent sanitary articles in a structure which is more comfortable and discrete and less obtrusive for the wearer and that can be worn in the wearer's usual undergarments.

The absorbent sanitary article according to the present invention is particularly rapid in acquiring a liquid surge, typical of incontinent adults, and has a multiple layer absorbent structure which is especially efficient in acquiring, distributing, and storing liquid exudates as they are deposited on the absorbent article.

The functions of acquiring and distributing exudates are handled by the acquisition and diffusion layer 60 which is separate from the fluff absorbent core 46 and the fluff-free absorbent core 54 which are used for storing the exudates.

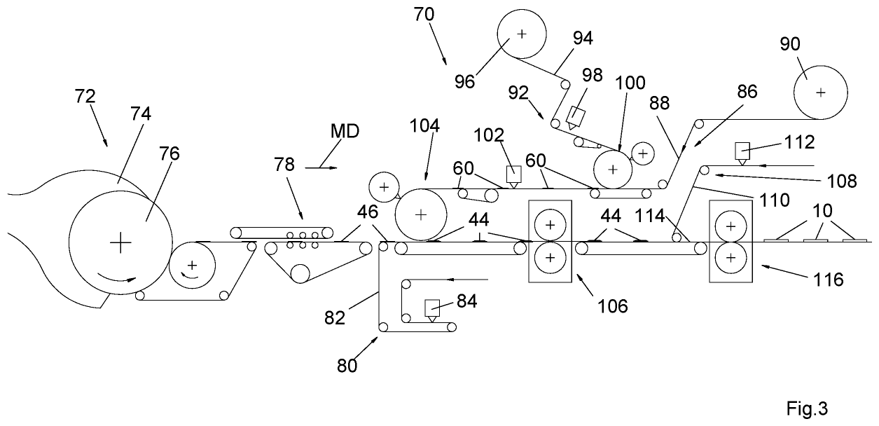

The absorbent sanitary articles 10 according to the present invention may be manufactured by a machine schematically shown in FIG. 3 and indicated by the reference numeral 70.

The machine 70 comprises a forming unit 72 comprising a stationary forming chamber 74 and a rotating forming wheel 76. The forming chamber 74 contains a mix of cellulose fluff and superabsorbent granular material dispersed in the cellulose fluff. On the outer surface of the forming wheel 76 a plurality of fluff absorbent cores 46 are formed, each comprising a mix of cellulose fluff and superabsorbent granular material and each having at least one through aperture, e.g. a channel shaped through aperture. A compacting conveyor 78 compacts and transports the fluff absorbent cores 46 in a machine direction MD. The fluff absorbent cores 46 are spaced apart from each other in the machine direction MD.

A first web feeding device 80 feeds a continuous topsheet web 82 in the machine direction MD. A first glue dispenser 84 applies a first glue layer on an upper surface of the continuous topsheet web 82.

The individual absorbent cores 46 moving in the machine direction MD are applied on the upper surface of the continuous topsheet web 82 and are attached thereto by the first glue layer.

A second web feeding device 86 feeds a continuous fluff-free absorbent non-woven web 88, comprising superabsorbent granular material dispersed in the non-woven web. The continuous fluff-free absorbent non-woven web 88 may be unwound from a reel 90.

In a possible embodiment, the continuous fluff-free absorbent non-woven web 88 may be manufactured in-line with the machine 70 by a method comprising:
depositing on a movable surface loose unbound fibres and granular superabsorbent material dispersed in the loose unbound fibres, and
binding and/or bonding together the loose unbound fibres so as to form an absorbent structure of bound fibres in which granular superabsorbent material is incorporated, where binding involves the use of binders such as glues or similar binding agents and bonding involves only the use of heat, pressure, air, etc. Such method is disclosed in detail in IT102020000019960 by the same Applicant.

A third web feeding device 92 feeds a continuous acquisition and diffusion web 94. The continuous acquisition and diffusion web 94 may be unwound from a reel 96. A second glue dispenser 98 applies a second glue layer on an upper surface of the continuous acquisition and diffusion web 94.

A first cut-and-slip unit 100 transversally cuts the continuous acquisition and diffusion web 94 to form individual acquisition and diffusion layers 60. The individual acquisition and diffusion layers 60 are spaced apart from each other in the direction of movement and are applied on the upper surface of the continuous fluff-free absorbent non-woven web 88 and are attached thereto by the second glue layer.

A third glue dispenser 102 applies a third glue layer on an upper surface of the continuous fluff-free absorbent non-woven web 88.

A second cut-and-slip unit 104 transversally cuts the continuous fluff-free absorbent non-woven web 88 along transverse cutting lines set between each pair of adjacent acquisition and diffusion layers 60, so as to form individual fluff-free absorbent cores 54 each having a respective acquisition and diffusion layer 60 applied thereon. The individual fluff-free absorbent cores 54 are spaced apart from each other in the machine direction MD and are applied over respective fluff absorbent cores 46 attached on the upper surface of the continuous topsheet web 80, as they move in the machine direction MD, so as to form an array of absorbent structures 44 each comprising an acquisition and diffusion layer 60 sandwiched between a respective fluff-free absorbent core 54 and a fluff absorbent core 46. The fluff-free absorbent cores 54 are attached to the respective fluff absorbent cores 46 by the third glue layer.

A compression unit 106 compresses the array of absorbent structures 44. The compression unit 106 may carry out an embossing of the continuous topsheet web 82 and press portions of the continuous topsheet web 82 inside the through apertures 52 of the fluff absorbent cores 46.

Pairs of side panels 30, 32 may be attached to the continuous topsheet web 82.

A fourth web feeding device 108 feeds a continuous backsheet web 110. A fourth glue dispenser 112 applies a fourth glue layer on an upper surface of the continuous backsheet web 110. The continuous backsheet web 110 is then overlapped to the continuous topsheet web 82. The array of absorbent structures 44 is sandwiched between the continuous topsheet web 82 and the continuous backsheet web 110, so as to form a continuous chassis chain 114.

A cutting unit 116 cuts the continuous chassis chain 114 so as to form individual absorbent sanitary articles 10.

A method for producing absorbent sanitary articles according to the present invention comprises:
providing a continuous topsheet web 82,
forming a plurality of fluff absorbent cores 46 comprising a mix of cellulose fluff and superabsorbent granular material dispersed in the cellulose fluff, wherein each of said fluff absorbent cores 46 has at least one through aperture 52,
applying said fluff absorbent cores 46 on the continuous topsheet web 82 spaced apart from each other in a machine direction MD,
providing a continuous fluff-free absorbent non-woven web 88 comprising superabsorbent granular material dispersed in the non-woven web,
providing a continuous acquisition and diffusion web 94,
transversely cutting the continuous acquisition and diffusion web 94 to form individual acquisition and diffusion layers 60,
spacing apart from each other in a longitudinal direction said individual acquisition and diffusion layers 60,
applying said individual acquisition and diffusion layers 60 on the continuous fluff-free absorbent non-woven web 88,
transversely cutting the continuous fluff-free absorbent non-woven web 88 so as to form individual fluff-free absorbent cores 54 each having a respective individual acquisition and diffusion layer 60 applied thereon,
spacing apart from each other in a longitudinal direction said individual fluff-free absorbent cores 54,
overlapping said individual fluff-free absorbent cores 54 to respective fluff absorbent cores 46 so as to form an array of absorbent structures 44 each comprising an acquisition and diffusion layer 60 sandwiched between a respective fluff-free absorbent core 54 and a fluff absorbent core 46, wherein the acquisition and diffusion layer 60 overlaps said at least one through aperture 52,
providing a continuous backsheet web 110,
applying the continuous backsheet web 110 over the continuous topsheet web 82 and sandwiching said array of absorbent structures 44 between said continuous topsheet web 82 and continuous backsheet web 110, so as to form a chassis chain 114, and
cutting said chassis chain 114 so as to form individual absorbent sanitary articles 10.

Of course, without prejudice to the principle of the invention, the details of construction and the embodiments can be varied, even significantly, with respect to those illustrated here without departing from the scope of the invention as defined by the following claims.

1. An absorbent sanitary article comprising a chassis including a liquid permeable topsheet, a liquid impermeable backsheet, and an absorbent structure arranged between the topsheet and the backsheet, wherein the absorbent structure comprises:
a fluff absorbent core comprising a mix of cellulose fluff and superabsorbent granular material dispersed in the mix of cellulose fluff, the fluff absorbent core being adjacent to the topsheet and having at least one through aperture,
a fluff-free absorbent core adjacent to the backsheet, comprising a non-woven web and superabsorbent granular material dispersed in the non-woven web, and
an acquisition and diffusion layer set between the fluff absorbent core and the fluff-free absorbent core and overlapping said at least one through aperture.

2. The absorbent sanitary article of claim 1, wherein said fluff absorbent core has a generally flat shape elongated in a longitudinal direction, and wherein said at least one through aperture has a shape of a channel elongated in said longitudinal direction.

3. The absorbent sanitary article of claim 1, wherein:
said fluff absorbent core has first and second opposite surfaces,
said fluff-free absorbent core has third and fourth opposite surfaces,
said acquisition and diffusion layer has fifth and sixth opposite surfaces, and
the first surface of the fluff absorbent core is in contact with the topsheet, the fourth surface of the fluff-free absorbent core is in contact with the backsheet, the fifth surface of the acquisition and diffusion layer is in contact with the second surface of the fluff absorbent core, and the sixth surface of the acquisition and diffusion layer is in contact with the third surface of the fluff-free absorbent core.

4. The absorbent sanitary article of claim 3, wherein the fifth surface of the acquisition and diffusion layer covers at least a portion of the second surface of the fluff absorbent core on which said at least one aperture extends.

5. The absorbent sanitary article of claim 3, wherein the sixth surface of the acquisition and diffusion layer is 30% or more of the third surface of the fluff-free absorbent core.

6. The absorbent sanitary article of claim 1, wherein said fluff-free absorbent core comprises super-absorbent granular material dispersed in the non-woven fibres in an amount comprised between 600 gsm.

7. The absorbent sanitary article of claim 1, wherein said fluff-free absorbent core comprises at least one layer of high loft non-woven material.

8. The absorbent sanitary article of claim 1, wherein said fluff-free absorbent core has a thickness comprised between 2 mm.

9. The absorbent sanitary article of claim 1, wherein said the fluff-free absorbent core comprises upper and lower non-woven layers.

10. The absorbent sanitary article of claim 9, wherein both upper and lower non-woven layers contain super-absorbent granular material.

11. The absorbent sanitary article of claim 9, wherein only the lower non-woven layer contains super-absorbent granular material.

12. The absorbent sanitary article of claim 1, wherein the fluff-free absorbent core is enclosed in a liquid permeable wrapping layer.

13. A method for producing absorbent sanitary articles, comprising:
providing a continuous topsheet web,
forming a plurality of fluff absorbent cores comprising a mix of cellulose fluff and superabsorbent granular material dispersed in the mix of cellulose fluff, wherein each of said plurality of fluff absorbent cores has at least one through aperture,
applying said plurality of fluff absorbent cores on the continuous topsheet web spaced apart from each other in a machine direction,
providing a continuous fluff-free absorbent non-woven web comprising superabsorbent granular material dispersed in the non-woven web,
providing a continuous acquisition and diffusion web,
transversely cutting the continuous acquisition and diffusion web to form individual acquisition and diffusion layers,
spacing apart from each other in a longitudinal direction said individual acquisition and diffusion layers,
applying said individual acquisition and diffusion layers on the continuous fluff-free absorbent non-woven web,
transversely cutting the continuous fluff-free absorbent non-woven web so as to form individual fluff-free absorbent cores each having a respective individual acquisition and diffusion layer applied thereon,
spacing apart from each other in a longitudinal direction said individual fluff-free absorbent cores,
overlapping said individual fluff-free absorbent cores to respective fluff absorbent cores so as to form an array of absorbent structures each comprising the acquisition and diffusion layer sandwiched between a respective fluff-free absorbent core and a fluff absorbent core, wherein the acquisition and diffusion layer overlaps said at least one through aperture,
providing a continuous backsheet web,
applying the continuous backsheet web over the continuous topsheet web and sandwiching said array of absorbent structures between said continuous topsheet web and continuous backsheet web so as to form a chassis chain, and
cutting said chassis chain so as to form individual absorbent sanitary articles.

14. The method of claim 13, comprising compressing said array of absorbent structures and pressing said continuous topsheet web into said at least one through apertures of said fluff absorbent cores.

15. The method of claim 13, comprising forming in-line said continuous fluff-free absorbent non-woven web by a method comprising:
depositing on a movable surface loose unbound fibres and granular superabsorbent material dispersed in said loose unbound fibres, and
binding and/or bonding together said loose unbound fibres so as to form an absorbent structure of bound fibres in which the granular superabsorbent material is incorporated.
